(19) United States
(12) Patent Application Publication
(10) Pub. No.: US 2002/0102601 A1
(43) Pub. Date: Aug. 1, 2002

(54) Mastitis assay

(76) Inventors: Julie Lydia Fitzpatrick, Glasgow (GB); Michael James Stear, Glasgow (GB); David Platt, Glasgow (GB); Karen Logan, Glasgow (GB)

Correspondence Address: MYERS BIGEL SIBLEY & SAJOVEC, PO BOX 37428, RALEIGH, NC, 27627, US

(21) Appl. No.: 10/109,378

(22) Filed: Mar. 27, 2002

Related U.S. Application Data
(63) Continuation of application No. 09/530,056, filed on Sep. 11, 2000, now abandoned.

(30) Foreign Application Priority Data
Oct. 21, 1997 (GB) 9722109.7
Oct. 21, 1998 (GB) PCT/GB98/03132

(51) Int. Cl. C12Q 1/68

(52) U.S. Cl. 435/6

(57) ABSTRACT

The present invention relates to a blood cell proliferation assay employing Staphylococcus aureus antigen capable of inducing a proliferative response. The assay finds particular application in testing cattle, including cows and bulls for use in predicting resistance to subclinical or clinical mastitis.

The present invention relates to an assay and method of testing cattle for use in predicting resistance to subclinical or clinical mastitis.

Mastitis is very costly to the dairy industry due to discarded milk, reduced yield, milk of lower value, increased rates of premature culling and occasional mortality (Esslemont and Spincer, 1993) and is recognised as one of the major diseases adversely affecting dairy cow welfare (Menzies, 1995). Strategies to improve mastitis resistance, rather than to simply manage the disease by antibiotic therapy and culling, are particularly timely in terms of both cow welfare and concern about antibiotic residues in meat and milk products.

Bulls and cows are selected for breeding purposes on their predicted genetic merit for milk yield, composition, and physical attributes (type). These predictions can use information from a variety of sources, from ancestors, an animal's own appearance or performance in the case of cows, but for bulls these predictions rely heavily on the performance of their daughters, which is normally obtained from a progeny testing programme.

In all major dairy breeding programmes, selection aims to increase milk production, while generally maintaining or perhaps slightly improving milk quality and type traits. However, selection based on such criteria are expected to result in an increased incidence of mastitis (Shook, 1989). Development of selection criteria based on the ability to resist diseases of economic and welfare importance, such as mastitis, are urgently needed.

A high somatic cell count SCC has been shown to indicate increased genetic susceptibility to mastitis, with genetic correlation estimates of the order of 0.6-0.8 (Mrode and Swanson, 1996). Breeding for reduced SCC is a long term strategy to reduce both herd call counts and to increase resistance to mastitis. The identification of bulls which are genetically superior for SCC is again achieved by progeny testing, from SCC test results on his daughters. Unfortunately, bulls are generally 6 or 7 years of age before this information is available. Greater progress could be achieved if bulls could be preselected for increased resistance to masistis before they enter the progeny testing programme.

It is among the objects of the present invention to obviate and/or mitigate at least one of the above identified disadvantages.

Broadly speaking the present invention is based on the use of Staphylococcus aureus antigen in an in vitro cell proliferation assay for predicting resistance to mastitis in cattle.

In a first aspect the present invention provides an in vitro assay for predicting resistance to mastitis in animals, the assay comprising:

a) mixing Staphylococaus aureus antigen capable of inducing a proliferative response in blood cells with a sample of blood obtained from an animal to be tested,

b) allowing cells in the sample of blood to proliferate, and

c) determining a level of proliferation of said cells, wherein the level of proliferation is predictive of said animal's resistance to mastitis.

The animals to be tested are typically cattle, including both cows and bulls. However the assay may be applicable to other commercial milk producing animals, such as goats and sheep.

Generally cells are isolated from the blood sample in order to be used in the assay. Typically isolated white blood cells may be utilised. More preferably peripheral blood mononuclear cells (PBM) may be used. The amount of blood cells used in the assay will generally be in the range 1×10⁴ to 1×10⁸, preferably 1×10⁵ to 1×10⁷, most preferably 1×10to 1×10.

It is understood that the term S. aureus antigen does not refer to any particular antigen and may include any/all S. aureus antigens capable of inducing a proliferative response. Typically whole S. aureus bacteria may serve as the antigen.

S. aureus is known to be a cause of clinical and subclinical mastitis in dairy cows and often results in persistent mastitis which chronic abcessation of the tissue, which results in lowered production and premature culling (Erskine et al., 1987; Wilson and Richards, 1980). It might therefore be expected that any pathogenic strain or S. aureus which may be isolated from cattle with subclinical or clinical mastitis may be used in the present assay. Surprisingly however, the present inventors have found that not all isolated strains of S. aureus give a desired result.

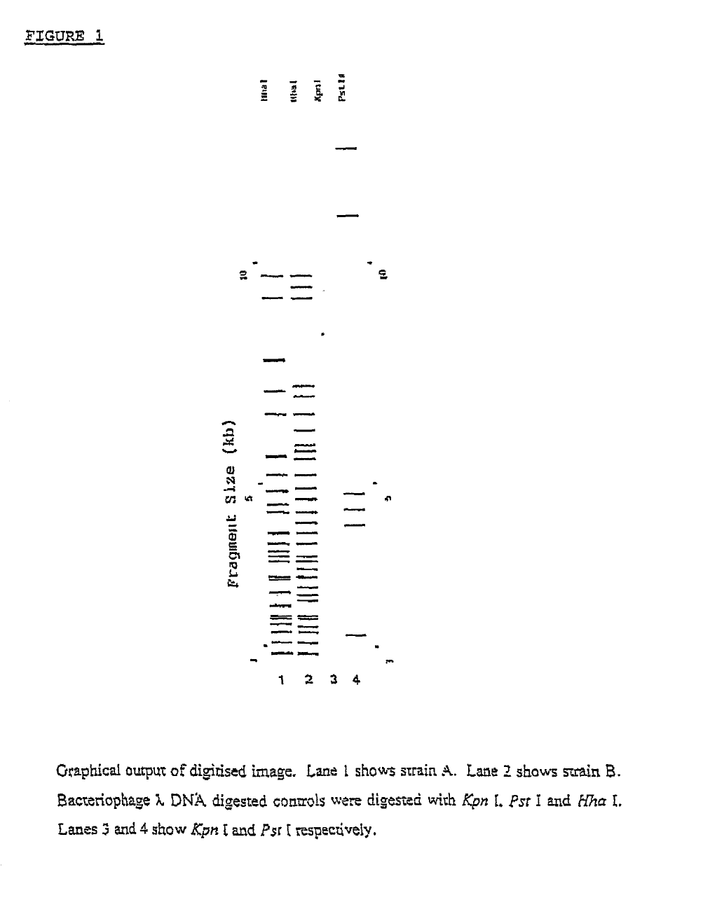

The present inventors have isolated a number of S. aureus strains from cattle with subclinical mastitis. The inventors were unable to readily distinguish the strains phenotypically and so the strains were distinguished on the basis of restriction fragmentation pattern analysis (REFP). Basically this technique involves isolating genomic DNA from a bacterial strain, digesting the DNA with a restriction enzyme and separating the restriction fragments by electrophoresis. As different strains are likely to have differences in their DNA, different restriction patterns are obtained. An example of the restriction fragment patterns of two strains (strains A and B) isolated by the inventors is shown in FIG. 1. Although no difference was observed between strains A and B at a phenotypic level, clear differences were observed at the DNA level.

The assay of the present invention has been performed using strains A and B and it has been found that Strain B does not elicit an immune response while strain A does. Strain A which may be identified by its restriction fragmentation pattern as shown in FIG. 1 is particularly preferred for use in the present invention. Strain A has been deposited in accordance with the Budapest Treaty with the National Collection of Type Cultures (NCTC) (Colindale, London, UK) on the Sep. 8, 1997 and is available under accession no. 13047.

Although it may be possible to perform the assay with live bacteria, it is generally desirable, particularly for safety reasons, to employ killed S. aureus. The killed S. aureus may is conveniently be formalin killed S. aureus. The amount of S. aureus cells used in the assay will generally be in the range 1×10³ to 1×10⁸, preferably 1×10⁴ to 1×10⁷, most preferably 1×10⁵ to 1×10⁶.

After contacting the blood cells with S. aureus, the blood cells are allowed to proliferate for a period of days, preferably between 5-15 days and a level of proliferation determined. Cell proliferation may be determined by a number of methods including addition of radio active nucleotides followed by scintillation counting.

Proliferation of the cells is used as a prediction of an animals resistance to mastitis. Cattle which show a high level of cell proliferation are predicted to be able to effectively control levels of S. aureus and consequently have a high level of resistance to mastitis. Conversely cattle which show a low level of cell proliferation are predicted to have a low level of resistance to mastitis. It has been found that the assay may be used to test bulls as well as cows. It is possible therefore to test bulls prior to a bull being used for breeding purposes in order to determine if the progeny of the bull are likely to have a low or high level of resistance to mastitis.

Generally, it may be desirable to have a standard from which it is possible to separate cattle into “high” and “low” resistance groups. The present inventors have expressed their results as what are termed “stimulation indices” (SI), which is essentially the level of proliferation of a culture with antigen (ie. S. aureus) divided by the level of proliferation without antigen. Generally speaking it is proposed that animals with an SI value of greater than 30, more preferably greater than 90 may be placed within a group considered as “high” resistance.

In time, information on response to this assay might be used in conjunction with other measures of susceptibility to mastitis, or genetic merit for SCC.

In a further aspect the present invention provides a test kit which may be used to test an animal in order to predict the animal's resistance to mastitis, the kit comprising, an S. aureus strain capable of inducing a proliferative response in blood cells of an animal to be tested.

A particularly preferred strain of S. aureus has been deposited in accordance with the Budapest Treaty at NCTC (Colindale, London, UK) on the Sep. 8, 1997 and is available under accession no. 13047.

The present invention will now be further described and understood with reference to the following examples section.

### EXAMPLE SECTION

### Example 1

Cell Isolation

Peripheral blood mononuclear cells (PBM) were isolated from peripheral blood by density centrifugation as follows:

Blood samples were collected from the jugular vein of cattle into sterile heparin vacutainers (Becton Dickenson). 10 mls of Hanks Balanced Salt Solution without calcium and magnesium (HBSS w/o Ca; Gibco) was added to 20 mls of the heparinised blood, which was then underlayed with 20 mls of ficollisopaque (Histopaque 1077 g/l; Sigma). Following centrifugation at 800 g for 40 mins at 20° C. the cells at the interface between the Histopaque and the media were carefully removed using a plastic pipette. The cells were resuspened in 50 mls of HBSS w/o Ca and Mg and then centrifuged at 600 g for 10 mins at 20° C., three times. The cell numbers were counted using White Cell Counting Fluid and adjusted to 4×10⁶/ml in complete media (Eagles BME (Gibco) supplemented with 2 mM L-glutamine (Gibco), 20 mM hepes (Gibco), 50 ug/50 units/ml streptomycin/penicillin (Gibco). Autologous serum was collected from each animal by blood sampling from the jugular vein into vacutainers with no anti-coagulant, incubating the tubes at 40° C. for 30 mins, 37° C. for 30 mins and then centrifuging the tubes at 800 g for 30 mins. The serum was collected by pipette and heat inactivated at 56° C. for 30 mins. 2% autologous serum was then added to the final cell suspension in complete media.

### Example 2

Antigen Preparation and Identification

Five S. aureus isolates were derived from S. aureus bacteria isolated from cases of subclinical bovine mastitis. The bacteria were initially cultured on Columbia blood agar. Ten milliliters of BHI broth (Oxoid) was inoculated aseptically from columbia agar culture slopes and incubated overnight at 37° C.

The S. aureus bacteria ware killed in 1% formalin for 18 hours at 4° C. and then washed four times by centrifugation at 800 g in sterile PBS for use in the proliferative assay. Inactivated bacteria were stored at −20° C.

The strains of S. aureus were identified by standard diagnostic methods and characterised by Restriction Enzyme Fragmentation Pattern Analysis (REFP) as follows:

Purification of DNA was carried out over a period of two days. On day one of the purification procedure, overnight BHI broths were centrifuged at 4480 g for 10 minutes and resuspended in 3 ml Tris ethylene diamine tetra acetic acid sodium chloride buffer (TES) (50 mM Tris-base, 50 mM sodium chloride, 5 mM disodium EDTA, (pH8.0). The samples were then aliquoted into 3 eppendorf tubes and microcentrifuged at 13,800 g for 30 seconds before being resuspended in 200 μl TES which contained 50 mM sucrose to prevent cell lysis. Twenty microliters of the endopeptidase lysostaphin (Sigma, 100 units/ml) which was specific for the pentaglycine bridge involved uniquely in the cell wall structure of S. aureus, and 100 μl lysozyme (Sigma, 40 mg/ml) were added and mixed thoroughly before being incubated at 37°0 C. for 30 minutes. After incubation 20% SDS (15 μl) was added and mixed by inversion, fifty microliters of Proteinase K (10 mg/ml) was also added before the sample was sheared through a 25 gauge needle to reduce the viscosity of the DNA and incubated for two hours at 37° C. One hundred microliters of TE₁₀ (10 mM Tris base, 10 mM disodium EDTA, (pH7.8) was added to reduce the DNA concentration before the initial purification stage using phenol chloroform (500 μl). After mixing thoroughly, the samples were microcentrifuged at 13,800 g for 10 minutes and the aqueous layer was transferred to another eppendorf and 500 μl of isopropanol was added. The samples were left at room temperature for 60 minutes to precipitate the DNA, followed by centrifugation at 13,800 g for 10 minutes. The pellet was resuspended in 100 μl TE₁₀ and triplicate tubes were pooled, 100 μl of ammonium acetate (7.5M, pH8.0) and 600 μl of 95% ethanol were added and samples were stored at −20° C. overnight, to precipitate the DNA.

On day two, further purification procedures were carried out. Samples were centrifuged at 13,800 g for 10 minutes, resuspended in 300 μl TE₁₀ and 20 μl RNAase (Sigma, 10 mg/ml) was added. After incubation at 37° C. for 60 minutes, the phenol/chloroform, isopropanol and ethanol steps were repeated as above and the samples were again stored at −20 overnight,

Restriction digestion was carried out on day three, the day immediately following the purification procedures. Samples were centrifuged at 13,800 g for 10 minutes and resuspended in 60 μl TE (10 mM Tris base, 1 mM disodium EDTA (pH8.0)). Restriction digestion of each sample using restriction enzyme HhaI (Gibco Life Technologies Limited, Paisley) was carried out, with bacteriophage λ DNA digested with HhaI, KpnI and PstI as controls. Restriction products mixed with loading buffer (25 g sucrose, 60 mg sodium acetate, 100 mg SDS and 50 mg bromophenol blue in 100 ml) were electrophoresed in a 0.8% agarose gel (2:1 dilution with TBE distilled water, (Tris base 90 mM, boric acid 89 mM disodium EDTA 1.25 mM, pH8.0-8.4) at 25 mA overnight and the gel was stained using ethidium bromide in TBE (0.5-1.0 ug/ml ethidium bromide). Following removal of excess background staining by rinsing the gel in distilled water for approximately 15 minutes, the gels were photographed on a transilluminator at 302 nm using Polaroid film type 655. REFP analysis identified differences at the DNA level between the five isolates. FIG. 1 shows a digitised representation of REFP results of two of the strains (strains A and B) where it can clearly be seen that there are differences in the restriction fragment patterns between the two strains.

### Example 3

Proliferative Assay

One ml of the cell suspension was added to each well of a 24 well, flat-bottomed tissue culture plate (Nunc) and one ml of bacterial suspension was also added to each well, giving final concentrations of 2×10cells/ml and 0.5×10bacteria/ml. The plates were incubated in a 5% CO₂ incubator at 37° C. for 10 days. 100 μl volumes of the cell suspension were removed and pulsed with 1 μCi of tritiate thymidine for 6 hours at 37° C. in 5% CO₂ incubator. Cells were harvested onto GFC bonded unifilter plates using a filtermat 196 and dried. Microscint O (Canberra Packard) was added and scintillation counting performed using a Topcount Microplate Scintillation and Luminescence Counter (Canberra Packard). Results were recorded as counts per minute (cpm) and expressed as stimulation indices (SI) which are cpm of cultures with antigen divided by the cpm of cultures with no antigen.

Statistical Analysis

The genetic merit of bulls (their Predicted Transmitting Abilities, or PTAs) for Somatic Cell Count (SCC) is calculated in the UK by the Animal Data Centre using procedures described by Mrode and Swanson (1996). These predictions use lactation averages for SCC on the daughters, after the individual test results have been transformed to natural logarithms. Averages for lactations 1-5 are used, assuming a heritability of 11%, and a repeatability across lactations of 35%. Other effects accounted for in the analysis include herd*year*season of calving, month and age of the cow at calving, and adjustments are also made for group effects which describe the breed, gender and country of origin of the ancestors.

The resulting proofs are expressed in percentage terms, which predict the percent change of the lactation average daughter performance for SCC, relative to the population average.

Two groups of cows were selected from a commercial dairy herd, each group being sired by a different bull. The immunological assay described above was performed on cells isolated from blood samples taken from the cows. Significant differences were detected between the mean proliferative response of the two groups, suggesting that immunological response and sire were correlated.

Two groups of bulls were selected as being the extreme five for high and low SCC PTAs, among these bulls owned by Genus Ltd which were still alive. The immunological assay described above was performed on cells isolated from blood samples taken from the bulls. After the data on their proliferative response were transformed to logarithms to account for the lack of normality of the data, there were significant differences between the two groups in response, indicating that immunological response and genetic merit for SCC are genetically correlated.

Results

The proliferative response was performed with two different strains of S. aureus. A digitised representative of strain A (which induced the immune response) and strain B (which did not induce the immune response) is shown in FIG. 1. Stain A has been deposited in accordance with the Budapest Treaty with NCTC on the Sep. 8, 1997 and is available under accession no. 13047.

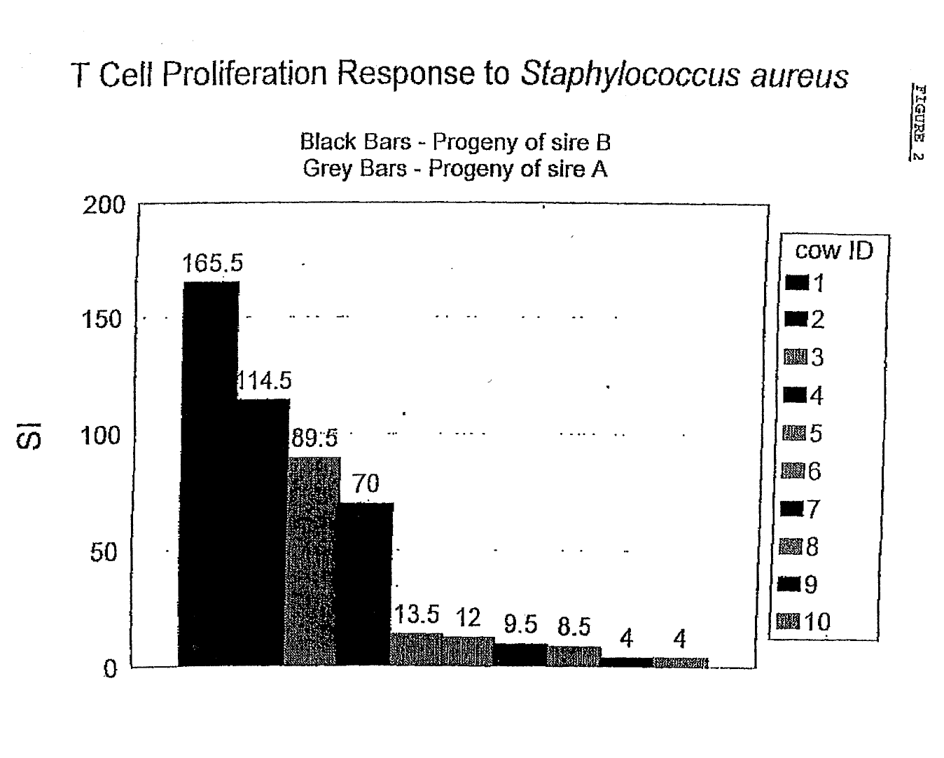

The proliferative assay was also performed on the two groups of cows and a significant difference between the two groups of cows was detected (p=0.032, FIG. 2).

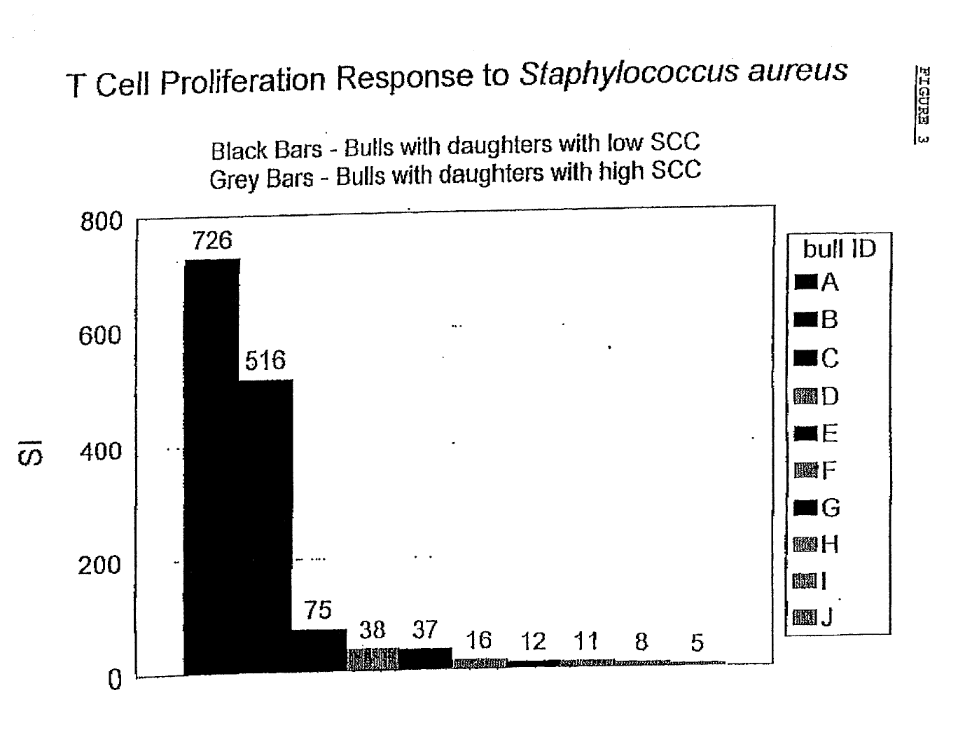

The proliferative assay was performed on five bulls with high PTA for SCC and five bulls with low PTA for SCC. A significant difference between the two groups of bulls (p<0.05) was detected and the regression of response on SCC proof was also significant (p,0.05; FIG. 3).

References

Erskine et al. (1987) Herd management and prevalence of mastitis in dairy herds with high and low somatic cell counts, JAVMA 190, 1411-1416.

Esslemont and Spincer, 1993 The incidence and costs of diseases in dairy herds. The Daisy Report no 2, Department of Agriculture, University of Reading.

Menzies et al. (1995) A study of mortality among suckler and dairy cows in Northern Ireland in 1992. Vet. Rec. 137, 531-536.

Mrode and Swanson, 1996 Genetic and statistical properties of somatic cell count and its suitability as an indirect means of reducing the incidence of mastitis in dairy cattle. Animal Breeding Abstracts 64, 847.

Shook (1989) J. Dairy Sci. 72, 1349.

1. An in vitro assay for predicting resistance to mastitis in animals, the assay comprising:
a) mixing Staphylococcus aureus antigen capable of inducing a proliferative response in blood cells, with a sample of blood obtained from an animal to be tested,
b) allowing cells in the sample of blood to proliferate, and
c) determining a level of proliferation of said cells, wherein the level of proliferation is predictive of said animal's resistance to mastitis.

2. The assay according to claim 1 wherein the sample of blood comprises isolated white blood cells.

3. The assay according to claim 2 wherein the isolated white blood cells are peripheral blood mononuclear (PBM) cells.

4. The assay according to any preceding claim wherein the amount of blood cells in the sample of blood is in the range 1×10⁴ to 1×10⁹.

5. The assay according to any preceding claim wherein the S. aureus antigen is a strain of S. aureus substantially displaying the restriction fragmentation pattern as shown in lane 1 of FIG. 1.

6. The assay according to claim 5 wherein the S. aureus strain is deposited in the National Collection of Type Cultures (NCTC) and is available under accession no. 13047.

7. The assay according to any preceding claim wherein the S. aureus is killed prior to use.

8. The assay according to any preceding claim wherein the proliferation is allowed to proceed for between 5-15 days.

9. The assay according to any preceding claim wherein the animals to be tested are cattle.

10. The assay according to claim 9 wherein the cattle are bulls.

11. A test kit for use in the assay according to any preceding claim wherein the kit comprises an S. aureus strain capable of inducing a proliferative response in blood cells of an animal to be tested.

12. A test kit according to claim 11 wherein the S. aureus strain is deposited it the NCTC and is available under accession no. 13047.

13. A method for predicting the suitability of cattle for breeding purposes wherein the animal is tested using the assay according to any one of claims 1 to 10 and wherein the level of cell proliferation is correlated with a level of resistance to mastitis.

14. The method according to claim 13 wherein the animal is a bull and the method is used as a prediction for determining if the progeny of the bull are likely to have a low or high level of resistance to mastitis.
